(19) United States
(12) Patent Application Publication
(10) Pub. No.: US 2022/0409579 A1
(43) Pub. Date: Dec. 29, 2022

(54) METHODS OF TREATING BINGE EATING DISORDER

(71) Applicant: OTSUKA AMERICA PHARMACEUTICAL, INC., Rockville, MD (US)

(72) Inventors: Anthony McKinney, Cambridge, MA (US); Randall Marshall, Cambridge, MA (US)

(21) Appl. No.: 17/693,067

(22) Filed: Mar. 11, 2022

Related U.S. Application Data
(63) Continuation of application No. 15/646,869, filed on Jul. 11, 2017, now abandoned.
(60) Provisional application No. 62/360,956, filed on Jul. 11, 2016.

(51) Int. Cl. A61K 31/403; A61K 45/06

(52) U.S. Cl. CPC A61K 31/403; A61K 45/06

(57) ABSTRACT

Provided is a method of treating binge eating disorder (BED) in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

This application claims priority to U.S. Provisional Application No. 62/360,956 filed Jul. 11, 2016, the contents of which are hereby incorporated by reference in entirety.

### BACKGROUND

Binge eating disorder (BED) is characterized by binge eating without purging, and is often, but not necessarily, associated with obesity. BED is challenging to treat and carries significant medical and psychological risks.

BED is associated with significant morbidity, including medical complications related to obesity (e.g., type 2 diabetes, cardiovascular disease, hypertension, stroke), eating disorder psychopathology (e.g., weight and shape concerns), psychiatric co-morbidity, reduced quality of life, and impaired social functioning. Psychiatric comorbidities that may occur with BED include depression, bipolar disorder, anxiety disorders, substance abuse disorders, and attention-deficit/hyperactivity disorder (ADHD).

Analysis of psychological and pharmacological treatments for BED found that psychotherapy and structured self-help treatments had more robust effects on outcomes, including binge abstinence. However, while specialized psychotherapies, such as cognitive behavior therapy and interpersonal therapy, and self-help strategies are effective for reducing binge eating, not all patients respond adequately and these treatments are generally not effective for the obesity associated with BED.

Further, some pharmacological treatments may cause a worsening of symptoms. For instance, antidepressants may cause a worsening of depression and weight gain. Meta-analysis of some placebo-controlled studies of antidepressants in patients with BED show significantly higher binge eating remission rates for the antidepressant group compared with the placebo group and no differences are found in the mean frequency of binge eating episodes at the end of treatment, in BMI, or in treatment discontinuation for any reason.

Accordingly, there exists a need for new treatments for BED.

### BRIEF SUMMARY

Provided is a method of treating binge eating disorder (BED) in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

Further areas of applicability of the present disclosure will become apparent from the detailed description provided hereinafter. It should be understood that the detailed description and specific examples, while indicating preferred embodiments, are intended for purposes of illustration only and are not intended to limit the scope of this disclosure.

### DETAILED DESCRIPTION

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

(1R,5S)-1-(naphthalen-2-yl)-3-azabicyclo[3.1.0]hexane, also known as (+)-1-(naphthalen-2-yl)-3-azabicyclo[3.1.0]hexane, is shown as Formula I below.

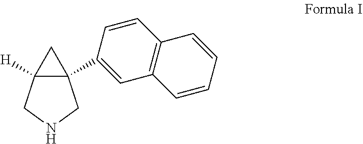 [SMILES: c1ccc2cc([C@]34CNC[C@H]3C4)ccc2c1]

“(1R,5S)-1-(naphthalen-2-yl)-3-azabicyclo[3.1.0]hexane” and “(+)-1-(naphthalen-2-yl)-3-azabicyclo[3.1.0]hexane” are used interchangeably herein.

(1R,5S)-1-(naphthalen-2-yl)-3-azabicyclo[3.1.0]hexane is an unbalanced triple reuptake inhibitor with the most potency towards norepinephrine reuptake (NE), one-sixth as much towards dopamine reuptake (DA), and one-fourteenth as much towards serotonin reuptake (5-HT).

While stimulants have been used in individuals with binge eating, such treatment may not be optimal because stimulants may result in increased anxiety and come with a risk of abuse, dependency, and diversion. The risk of increased anxiety and abuse and dependency for stimulants may be especially concerning with this patient population as anxiety disorders and substance abuse disorders are often comorbid with BED.

However, a drug with minimal to no effect on dopamine may not provide optimal treatment for binge eating disorder either.

(1R,5S)-1-(naphthalen-2-yl)-3-azabicyclo[3.1.0]hexane enhances dopamine function, but without the pronounced dopaminergic activities of stimulants. Thus, in individuals with BED, the unbalanced norepinephrine-dopamine-serotonin reuptake inhibition profile of (1R,5S)-1-(naphthalen-2-yl)-3-azabicyclo[3.1.0]hexane may affect the norepinephrine, dopamine, and serotonin circuitries without causing increased anxiety and irritability and without triggering substance abuse that may be seen with stimulants.

In addition, the multi-functional effects of (1R,5S)-1-(naphthalen-2-yl)-3-azabicyclo[3.1.0]hexane may be useful in treating BED as well as comorbidities, such as depression, bipolar disorder, anxiety disorders, substance abuse disorders, personality disorder, somatoform disorder, and attention-deficit/hyperactivity disorder.

Further, affecting the norepinephrine, dopamine, and serotonin circuitries with one drug may avoid pharmacologic drug-drug interactions (DDIs). For instance, administering two drugs with different effects on the norepinephrine, dopamine, and/or serotonin circuitries may result in off-target interactions potentially leading to undesirable and unexpected norepinephrine, dopamine, and/or serotonin levels.

It has been reported that cytochrome P450 enzyme isoforms (CYPs), which catalyze oxidative reactions, account for the metabolism of 75% of all drugs, and in particular, about 80% of drugs cleared by CYPs are metabolized by four CYP isoforms—CYP3A4, CYP2D6, CYP2C9 and CYP2C19. Thus, these four CYPs are potential candidates for DDIs. Physicians consider potential DDIs and metabolic pathway(s) when selecting treatments. (1R,5S)-1-(naphthalen-2-yl)-3-azabicyclo[3.1.0]hexane is primarily metabolized by monoamine oxidase A (MAO A), which may spare the liver and may reduce drug-drug interactions if (1R,5S)-1-(naphthalen-2-yl)-3-azabicyclo[3.1.0]hexane is used in a combination therapy, e.g., with a drug that is metabolized by CYPs.

(1R,5S)-1-(naphthalen-2-yl)-3-azabicyclo[3.1.0]hexane may be synthesized as described in U.S. Pat. No. 8,461,196, U.S. Patent Publication No. 2014/0206740, or International Publication No. WO 2013/019271, each of which are incorporated herein by reference in their entirety.

As used herein, “(1R,5S)-1-(naphthalen-2-yl)-3-azabicyclo[3.1.0]hexane” embraces the compound in any form, for example, free or pharmaceutically acceptable salt form, e.g., as a pharmaceutically acceptable acid addition salt. Pharmaceutically acceptable salts are known in the art and include salts that are physiologically acceptable at the dosage amount and form to be administered, for example, hydrochloride salts.

As used herein, “(1R,5S)-1-(naphthalen-2-yl)-3-azabicyclo[3.1.0]hexane” is also to be understood as embracing the compound in crystalline and amorphous form including, for example, polymorphs, solvates (including hydrates), unsolvated polymorphs (including anhydrates), conformational polymorphs, and amorphous forms of the compounds, as well as mixtures thereof. “Crystalline form” and “polymorph” may be used interchangeably herein, and are meant to include all crystalline forms of (1R,5S)-1-(naphthalen-2-yl)-3-azabicyclo[3.1.0]hexane, in free or pharmaceutically acceptable salt form, including, for example, polymorphs, solvates (including hydrates), unsolvated polymorphs (including anhydrates), and conformational polymorphs, as well as mixtures thereof, unless a particular crystalline form is referred to.

Crystalline and amorphous forms of (1R,5S)-1-(naphthalen-2-yl)-3-azabicyclo[3.1.0]hexane may be used in any combination or in forms that are substantially free of one or more of the other crystalline forms or free of the amorphous form.

As used herein, “substantially free of other polymorphic forms” means that the crystalline material contains no more than 10% w/w of any other crystalline form, e.g., no more than 5% w/w of any other crystalline form, e.g., no more than 2% w/w of any other crystalline form, e.g., no more than 1% w/w of any other crystalline form.

(1R,5S)-1-(naphthalen-2-yl)-3-azabicyclo[3.1.0]hexane may in some cases also exist in prodrug form. Prodrugs are considered to be any covalently bonded carriers that release the active parent drug in vivo.

As used herein, the term “patient” includes human or non-human (i.e., animal) patient. In some embodiments, patient encompasses both human and nonhuman. In some embodiments, patient means a nonhuman. In other embodiments, patient means a human.

As used herein, “concurrently” means the compounds are administered simultaneously or within the same composition. In some embodiments, the compounds are administered simultaneously. In some embodiments, the compounds are administered within the same composition.

Anxiety may be characterized by feelings of tension and worried thoughts. Anxiety disorders may cause recurring intrusive thoughts or concerns. As used herein, “anxiety disorder” includes generalized anxiety disorder, panic disorder, social anxiety disorder, obsessive-compulsive disorder, phobias, and post-traumatic stress disorder.

As used herein, “substance abuse disorder” includes alcohol-related disorders, nicotine-related disorders, amphetamine-related disorders, cannabis-related disorders, cocaine-related disorders, hallucinogen-use disorders, inhalant-related disorders, and opioid-related disorders. For instance, “substance abuse disorder” includes alcohol abuse, nicotine abuse, opioid abuse, and stimulant (like cocaine and methamphetamine) abuse.

As used herein, “somatoform disorder” includes somatization disorder, conversion disorder, pain disorder, hypochondriasis, body dysmorphic disorder, undifferentiated somatoform disorder, and somatoform disorder not otherwise specified (NOS).

As used herein, “therapeutically effective amount” refers to an amount effective, when administered to a human or non-human patient, to provide a therapeutic benefit, such as amelioration of symptoms or elimination of the disease. The specific dose of substance administered to obtain a therapeutic benefit will, of course, be determined by the particular circumstances surrounding the case, including, for example, the specific substance administered, the route of administration, the condition being treated, and the individual being treated.

As used herein, “treatment” and “treating” encompass amelioration of symptoms or elimination of binge eating disorder, as well as treatment of the cause of binge eating disorder. For example, “treatment” and “treating” encompass suppression or improvement of the symptoms of binge eating disorder.

Binge eating disorder involves recurrent episodes of binge eating. A binge-eating episode may encompass eating, in a discrete period of time (e.g., within any 2-hour period), an amount of food that is larger than most people would eat during a similar period of time and under similar circumstances and a sense of lack of control over eating during the episode (e.g., a feeling that one cannot stop eating or control what or how much one is eating). A binge-eating episode may also encompass three (or more) of the following: eating much more rapidly than normal, eating until feeling uncomfortably full, eating large amounts of food when not feeling physically hungry, eating alone because of being embarrassed by how much one is eating, and feeling disgusted with oneself, depressed, or very guilty after overeating. Binge eating disorder may also encompass marked distress regarding binge eating. Binge eating may occur, on average, at least once a week for three months. Binge eating is not associated with the recurrent use of inappropriate compensatory behavior (for example, purging).

Provided is a method (Method 1) of treating binge eating disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

Further provided is a method (Method 2) of treating comorbid binge eating disorder and another psychiatric disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient, e.g., further provided is
i. a method (Method 2i) of treating comorbid binge eating disorder and an anxiety disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
ii. a method (Method 2ii) of treating comorbid binge eating disorder and a substance abuse disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
iii. a method (Method 2iii) of treating comorbid binge eating disorder and depression in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
iv. a method (Method 2iv) of treating comorbid binge eating disorder and bipolar disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
v. a method (Method 2v) of treating comorbid binge eating disorder and a personality disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
vi. a method (Method 2vi) of treating comorbid binge eating disorder and a somatoform disorder (e.g., body dysmorphic disorder) in a patient in need thereof comprising administering a therapeutically effective amount of (1R,5S)-1-(naphthalen-2-yl)-3-azabicyclo[3.1.0]hexane, in free or pharmaceutically acceptable salt form, to the patient; or
vii. a method (Method 2vii) of treating comorbid binge eating disorder and attention-deficit/hyperactivity disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

Further provided is a method (Method 3) of treating comorbid binge eating disorder and obesity in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

Further provided is a method (Method 4) of treating comorbid binge eating disorder and fibromyalgia in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

Further provided is any of Method 1, 2, 3, or 4, e.g., Method 1, e.g., Method 2 (e.g., any of Method 2i, Method 2ii, Method 2iii, Method 2iv, Method 2v, Method 2vi, or Method 2vii), e.g., Method 3, e.g., Method 4, as follows:
1.1 Any of Method 1, 2, (e.g., any of Method 2i, Method 2ii, Method 2iii, Method 2iv, Method 2v, Method 2vi, or Method 2vii), 3, or 4 wherein (1R,5S)-1-(naphthalen-2-yl)-3-azabicyclo[3.1.0]hexane is in pharmaceutically acceptable salt form.
1.2 Method 1.1 wherein (1R,5S)-1-(naphthalen-2-yl)-3-azabicyclo[3.1.0]hexane in pharmaceutically acceptable salt form is an acid addition salt.
1.3 Method 1.2 wherein (1R,5S)-1-(naphthalen-2-yl)-3-azabicyclo[3.1.0]hexane in pharmaceutically acceptable salt form is (1R,5S)-1-(naphthalen-2-yl)-3-azabicyclo[3.1.0]hexane hydrochloride.
1.4 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is crystalline.
1.5 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered at a dosage of 1 mg to 1800 mg, e.g., 10 mg to 1800 mg, e.g., 25 mg to 1800 mg, e.g., 10 mg to 1600 mg, e.g., 10 mg to 1200 mg, e.g., 50 mg to 1200 mg, e.g., 50 mg to 1000 mg, e.g., 75 mg to 1000 mg, e.g., 75 mg to 800 mg, e.g., 75 mg to 500 mg, e.g., 100 mg to 750 mg, e.g., 100 mg to 500 mg, e.g., 100 mg to 400 mg, e.g., 100 mg to 300 mg, e.g., 100 mg to 200 mg.
1.6 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered at a dosage of 75 mg to 1000 mg, e.g., 100 mg to 600 mg, e.g., 100 mg to 400 mg, e.g., 100 mg to 200 mg.
1.7 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered at a dosage of 50 mg to 600 mg, e.g., 100 mg to 600 mg, e.g., 100 mg to 400 mg, e.g., 100 mg to 200 mg, e.g., 100 mg, e.g., 200 mg, e.g., 400 mg.
1.8 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered at a dosage of 5 mg to 500 mg, e.g., 5 mg to 10 mg, e.g., 10 mg to 25 mg, e.g., 30 mg to 50 mg, e.g., 10 mg to 300 mg, e.g., 25 mg to 300 mg, e.g., 50 mg to 100 mg, e.g., 100 mg to 250 mg, e.g., 250 mg to 500 mg.
1.9 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered at a dosage of 0.5 mg/kg to 20 mg/kg per day, e.g., 1 mg/kg to 15 mg/kg per day, e.g., 1 mg/kg to 10 mg/kg per day, e.g., 2 mg/kg to 20 mg/kg per day, e.g., 2 mg/kg to 10 mg/kg per day, e.g., 3 mg/kg to 15 mg/kg per day.
1.10 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered once, twice, three, or four times daily.
1.11 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered concurrently or sequentially, in either order, with at least one other therapeutic agent. (1R,5S)-1-(naphthalen-2-yl)-3-azabicyclo[3.1.0]hexane, in free or pharmaceutically acceptable salt form, and the at least one other therapeutic agent may be combined in a single composition or administered in different compositions.
1.12 Method 1.11 wherein the other therapeutic agent is selected from an anti-depressant (e.g., a selective serotonin reuptake inhibitor (SSRI), a serotonin-norepinephrine reuptake inhibitor (SNRI), or a tricyclic anti-depressant), an anxiolytic, an anti-obesity agent, and an attention-deficit/hyperactivity disorder agent.
1.13 Method 1.11 wherein the other therapeutic agent is a second agent that is capable of treating one or more symptoms of binge eating disorder or conditions associated with binge eating disorder (e.g., type 2 diabetes, cardiovascular disease, high blood pressure, high cholesterol and/or triglycerides, kidney disease, gallbladder disease, arthritis, bone deterioration, stroke, upper respiratory infection, skin disorders, menstrual irregularities, ovarian abnormalities, pregnancy complications).
1.14 Any of Methods 1, 2 (e.g., any of Method 2i, Method 2ii, Method 2iii, Method 2iv, Method 2v, Method 2vi, or Method 2vii), 3, or 4 et seq., wherein the patient further undergoes at least one of cognitive behavioral therapy (CBT) (e.g., guided self-help CBT), interpersonal psychotherapy (IPT), behavioral weight loss treatment (BWL), or dialectical behavior therapy (DBT).
1.15 Any of Methods 1, 2 (e.g., any of Method 2i, Method 2ii, Method 2iii, Method 2iv, Method 2v, Method 2vi, or Method 2vii), 3, or 4 et seq., wherein (1R,5S)-1-(naphthalen-2-yl)-3-azabicyclo[3.1.0]hexane, in free or pharmaceutically acceptable salt form, is administered in a sustained release pharmaceutical composition, e.g., a composition as described in International Publication No. WO 2015/102826, which also published as U.S. Patent Publication No. 2016/0303077, both of which are hereby incorporated by reference in their entireties.
1.16 Any of Methods 1, 2 (e.g., any of Method 2i, Method 2ii, Method 2iii, Method 2iv, Method 2v, Method 2vi, or Method 2vii), 3, or 4 et seq., wherein the binge eating disorder comprises 1-3 binge episodes weekly.
1.17 Any of Methods 1, 2 (e.g., any of Method 2i, Method 2ii, Method 2iii, Method 2iv, Method 2v, Method 2vi, or Method 2vii), 3, or 4 et seq., wherein the binge eating disorder comprises 4-7 binge episodes weekly.
1.18 Any of Methods 1, 2 (e.g., any of Method 2i, Method 2ii, Method 2iii, Method 2iv, Method 2v, Method 2vi, or Method 2vii), 3, or 4 et seq., wherein the binge eating disorder comprises 8-13 binge episodes weekly.
1.19 Any of Methods 1, 2 (e.g., any of Method 2i, Method 2ii, Method 2iii, Method 2iv, Method 2v, Method 2vi, or Method 2vii), 3, or 4 et seq., wherein the binge eating disorder comprises 14 or more binge episodes weekly.

Also provided is use of (1R,5S)-1-(naphthalen-2-yl)-3-azabicyclo[3.1.0]hexane, in free or pharmaceutically acceptable salt form, in the manufacture of a medicament for treating binge eating disorder, e.g., for use in any of Method 1 or 1.1-1.19.

Also provided is use of (1R,5S)-1-(naphthalen-2-yl)-3-azabicyclo[3.1.0]hexane, in free or pharmaceutically acceptable salt form, in the manufacture of a medicament for treating comorbid binge eating disorder and another psychiatric disorder, e.g., for use in any of Method 2 (e.g., any of Method 2i, Method 2ii, Method 2iii, Method 2iv, Method 2v, Method 2vi, or Method 2vii) or 1.1-1.19.

Also provided is use of (1R,5S)-1-(naphthalen-2-yl)-3-azabicyclo[3.1.0]hexane, in free or pharmaceutically acceptable salt form, in the manufacture of a medicament for treating comorbid binge eating disorder and obesity, e.g., for use in any of Method 3 or 1.1-1.19.

Also provided is use of (1R,5S)-1-(naphthalen-2-yl)-3-azabicyclo[3.1.0]hexane, in free or pharmaceutically acceptable salt form, in the manufacture of a medicament for treating comorbid binge eating disorder and fibromyalgia, e.g., for use in any of Method 4 or 1.1-1.19.

Also provided is a pharmaceutical composition comprising (1R,5S)-1-(naphthalen-2-yl)-3-azabicyclo[3.1.0]hexane, in free or pharmaceutically acceptable salt form, in combination with a pharmaceutically acceptable diluent or carrier for use in treating binge eating disorder, e.g., for use in any of Method 1 or 1.1-1.19.

Also provided is a pharmaceutical composition comprising (1R,5S)-1-(naphthalen-2-yl)-3-azabicyclo[3.1.0]hexane, in free or pharmaceutically acceptable salt form, in combination with a pharmaceutically acceptable diluent or carrier for use in treating comorbid binge eating disorder and another psychiatric disorder, e.g., for use in any of Method 2 (e.g., any of Method 2i, Method 2ii, Method 2iii, Method 2iv, Method 2v, Method 2vi, or Method 2vii) or 1.1-1.19.

Also provided is a pharmaceutical composition comprising (1R,5S)-1-(naphthalen-2-yl)-3-azabicyclo[3.1.0]hexane, in free or pharmaceutically acceptable salt form, in combination with a pharmaceutically acceptable diluent or carrier for use in treating comorbid binge eating disorder and obesity, e.g., for use in any of Method 3 or 1.1-1.19.

Also provided is a pharmaceutical composition comprising (1R,5S)-1-(naphthalen-2-yl)-3-azabicyclo[3.1.0]hexane, in free or pharmaceutically acceptable salt form, in combination with a pharmaceutically acceptable diluent or carrier for use in treating comorbid binge eating disorder and fibromyalgia, e.g., for use in any of Method 4 or 1.1-1.19.

A dose or method of administration of the dose of the present disclosure is not particularly limited. Dosages employed in practicing the present disclosure will of course vary depending, e.g. on the mode of administration and the therapy desired. In general, satisfactory results, e.g. for the treatment of BED are indicated to be obtained on oral administration at dosages of the order from about 0.01 to 2.0 mg/kg. An indicated daily dosage for oral administration may be in the range of from about 0.75 mg to 200 mg, conveniently administered once, or in divided doses 2 to 4 times, daily or in sustained release form. Unit dosage forms for oral administration thus for example may comprise from about 0.2 mg to 75 mg or 150 mg, e.g. from about 0.2 mg or 2.0 mg or 50 mg or 75 mg or 100 mg to 200 mg or 500 mg of (1R,5S)-1-(naphthalen-2-yl)-3-azabicyclo[3.1.0]hexane, in free or pharmaceutically acceptable salt form, together with a pharmaceutically acceptable diluent or carrier therefor.

(1R,5S)-1-(naphthalen-2-yl)-3-azabicyclo[3.1.0]hexane, in free or pharmaceutically acceptable salt form, may be administered by any suitable route, including orally, parenterally, transdermally, or by inhalation, including by sustained release, although various other known delivery routes, devices and methods can likewise be employed. In some embodiments, (1R,5S)-1-(naphthalen-2-yl)-3-azabicyclo[3.1.0]hexane, in free or pharmaceutically acceptable salt form, is administered in a sustained release pharmaceutical composition, e.g., an oral sustained release pharmaceutical composition, comprising (1R,5S)-1-(naphthalen-2-yl)-3-azabicyclo[3.1.0]hexane, in free or pharmaceutically acceptable salt form, which provides therapeutically effective levels of (1R,5S)-1-(naphthalen-2-yl)-3-azabicyclo[3.1.0]hexane over a sustained delivery period of approximately 6 hours or longer, e.g., 8 hours or longer, e.g., 12 hours or longer, e.g., 18 hours or longer, e.g., 24 hours or longer. In some embodiments, (1R,5S)-1-(naphthalen-2-yl)-3-azabicyclo[3.1.0]hexane, in free or pharmaceutically acceptable salt form, is administered in an immediate release pharmaceutical composition, e.g., an oral immediate release pharmaceutical composition, comprising (1R,5S)-1-(naphthalen-2-yl)-3-azabicyclo[3.1.0]hexane, in free or pharmaceutically acceptable salt form.

Pharmaceutical compositions comprising (1R,5S)-1-(naphthalen-2-yl)-3-azabicyclo[3.1.0]hexane, in free or pharmaceutically acceptable salt form, may be prepared using conventional diluents or excipients and techniques known in the galenic art. Thus, oral dosage forms may include tablets, capsules, solutions, suspensions, and the like.

Where two active agents are administered, the therapeutically effective amount of each agent may be below the amount needed for activity as monotherapy. Accordingly, a subthreshold amount (i.e., an amount below the level necessary for efficacy as monotherapy) may be considered therapeutically effective. Indeed, an advantage of administering different agents with different mechanisms of action and different side effect profiles may be to reduce the dosage and side effects of either or both agents, as well as to enhance or potentiate their activity as monotherapy.

1. A method of treating binge eating disorder in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

2. The method of claim 1, further comprising treating another psychiatric disorder, or obesity, or fibromyalgia, in a patient in need thereof by said administering a therapeutically effective amount of (1R,5S)-1-(naphthalen-2-yl)-3-azabicyclo[3.1.0]hexane, in free or pharmaceutically acceptable salt form, to the patient.

3. The method of claim 2, wherein the other psychiatric disorder is an anxiety disorder, a substance abuse disorder, bipolar disorder, a personality disorder, a somatoform disorder, or attention deficit/hyperactivity disorder.

4. The method of claim 2, comprising treating comorbid binge eating disorder and obesity in a patient in need thereof comprising administering a therapeutically effective amount of (1R,5S)-1-(naphthalen-2-yl)-3-azabicyclo[3.1.0]hexane, in free or pharmaceutically acceptable salt form, to the patient.

5. (canceled)

6. The method of claim 1, wherein (1R,5S)-1-(naphthalen-2-yl)-3-azabicyclo[3.1.0]hexane is in pharmaceutically acceptable salt form.

7. (canceled)

8. The method of claim 1, wherein (1R,5S)-1-(naphthalen-2-yl)-3-azabicyclo[3.1.0]hexane in pharmaceutically acceptable salt form is (1R,5S)-1-(naphthalen-2-yl)-3-azabicyclo[3.1.0]hexane hydrochloride.

9. The method of claim 1, wherein (1R,5S)-1-(naphthalen-2-yl)-3-azabicyclo[3.1.0]hexane, in free or pharmaceutically acceptable salt form, is administered at a dosage of 1 mg to 1800 mg, or 10 mg to 1800 mg, or 25 mg to 1800 mg, or 10 mg to 1600 mg, or 10 mg to 1200 mg, or 50 mg to 1200 mg, or 50 mg to 1000 mg, or 75 mg to 1000 mg, or 75 mg to 800 mg, or 75 mg to 500 mg, or 100 mg to 750 mg, or 100 mg to 500 mg, or 100 mg to 400 mg, or 100 mg to 300 mg, or 100 mg to 200 mg.

10. (canceled)

11. (canceled)

12. (canceled)

13. The method of claim 1, wherein (1R,5S)-1-(naphthalen-2-yl)-3-azabicyclo[3.1.0]hexane, in free or pharmaceutically acceptable salt form, is administered at a dosage of 0.5 mg/kg to 20 mg/kg per day, or 1 mg/kg to 15 mg/kg per day, or 1 mg/kg to 10 mg/kg per day, or 2 mg/kg to 20 mg/kg per day, or 2 mg/kg to 10 mg/kg per day, or 3 mg/kg to 15 mg/kg per day.

14. The method of claim 1, wherein (1R,5S)-1-(naphthalen-2-yl)-3-azabicyclo[3.1.0]hexane, in free or pharmaceutically acceptable salt form, is administered once, twice, three, or four times daily.

15. The method of claim 1, wherein (1R,5S)-1-(naphthalen-2-yl)-3-azabicyclo[3.1.0]hexane, in free or pharmaceutically acceptable salt form, is administered concurrently or sequentially, in either order, with at least one other therapeutic agent.

16. The method of claim 15, wherein the other therapeutic agent is selected from an anti-depressant, wherein the anti-depressant comprises one or more in the group of a selective serotonin reuptake inhibitor (SSRI), a serotonin-norepinephrine reuptake inhibitor (SNRI), or a tricyclic anti-depressant, an anxiolytic, an anti-obesity agent, and an attention-deficit/hyperactivity disorder agent.

17. The method of claim 15, wherein the other therapeutic agent is a second agent that is capable of treating one or more symptoms of binge eating disorder or conditions associated with binge eating disorder, wherein the symptoms and/or conditions are selected from type 2 diabetes, cardiovascular disease, high blood pressure, high cholesterol and/or triglycerides, kidney disease, gallbladder disease, arthritis, bone deterioration, stroke, upper respiratory infection, skin disorders, menstrual irregularities, ovarian abnormalities, and pregnancy complications.

18. The method of claim 1, wherein the patient further undergoes at least one of cognitive behavioral therapy (CBT), guided self-help CBT, interpersonal psychotherapy (IPT), behavioral weight loss treatment (BWL), or dialectical behavior therapy (DBT).

19. The method of claim 1, wherein the binge eating disorder comprises 1-3 binge episodes weekly.

20. The method of any one of claim 1, wherein the binge eating disorder comprises 4-7 binge episodes weekly.

21. The method of claim 1, wherein the binge eating disorder comprises 8-13 binge episodes weekly.

22. The method of claim 1, wherein the binge eating disorder comprises 14 or more binge episodes weekly.

23. The method of claim 15, wherein the therapeutically effective amount of (1R,5S)-1-(naphthalen-2-yl)-3-azabicyclo[3.1.0]hexane, in free or pharmaceutically acceptable salt form, is the sole compound administered which can affect the norepinephrine, dopamine, and serotonin circuitries.

24. The method of claim 15, wherein the at least one other therapeutic agent is primarily metabolized by one or more CYPs rather than monoamine oxidase A.

25. The method of claim 1, wherein the at least one other therapeutic is not primarily metabolized by monoamine oxidase A.
